(19) United States
(12) Patent Application Publication
(10) Pub. No.: US 2024/0225557 A1
(43) Pub. Date: Jul. 11, 2024

(54) ACCELERATION BASED SHALLOW BREATHING RISK FACTOR

(71) Applicant: Cardiac Pacemakers, Inc., St. Paul, MN (US)

(72) Inventors: Jonathan Bennett Shute, Eagan, MN (US); Bin Mi, Arden Hills, MN (US); John D. Hatlestad, Maplewood, MN (US); Kevin Chu, Shoreview, MN (US); Viktoria A. Averina, Shoreview, MN (US)

(21) Appl. No.: 18/391,571

(22) Filed: Dec. 20, 2023

Related U.S. Application Data
(60) Provisional application No. 63/438,060, filed on Jan. 10, 2023.

(51) Int. Cl. A61B 5/00; A61B 5/11; A61B 5/113

(52) U.S. Cl. CPC A61B 5/7275; A61B 5/1116; A61B 5/1118; A61B 5/113; A61B 5/6823; A61B 5/7264; A61B 5/7282; A61B 2560/0223; A61B 2562/0219; A61N 1/36031

(57) ABSTRACT

Systems and methods for recognizing and classifying breathing patterns based on spatial analysis of chest wall or abdominal movement or acceleration are described. A medical-device system comprises a receiver circuit to receive respiration information of a patient, and a breath analyzer circuit to determine from the respiration information two or more spatial respiration components, such as accelerations of chest wall or abdominal movement in respective directions along the anatomical axes. The breath analyzer circuit can classify a breathing pattern as one of either chest breathing or diaphragmatic breathing based on the determined spatial respiration components. The classified breathing pattern can be used for detecting a physiological event such as worsening heart failure or for assessing the patient's risk of having metabolic disorders.

### CLAIM OF PRIORITY

This application claims the benefit of U.S. Provisional Application No. 63/438,060, filed on Jan. 10, 2023, which is hereby incorporated by reference in its entirety.

### TECHNICAL FIELD

This document relates generally to medical devices, and more particularly, to systems, devices and methods for recognizing a breathing pattern of a subject and stratifying shallow breathing risk and detecting a respiratory or cardiac event based on the breathing pattern.

### BACKGROUND

Breathing is an automatic unconscious process, predominantly consisting of active inspiration and passive expiration. Different muscles are involved in the breathing process. Diaphragm is a large muscle that sits at the base of the lungs. During inspiration, the diaphragm contracts and flattens, and the chest cavity enlarges. This contraction creates a vacuum that pulls air into the lungs. During expiration, the diaphragm relaxes and returns to its normal shape, and air is forced out of the lungs.

Breathing can be shallow or deep. Normal breathing is generally relatively shallow and does not use the full capacity of the lungs. Chest breathing (also known as shallow breathing, thoracic breathing, or costal breathing) is the drawing of minimal breath into the lungs using the muscles in the upper chest and rib cage (intercostal muscles) instead of the diaphragm. Diaphragmatic breathing (also known as deep breathing, belly breathing, or abdominal breathing) typically fully engages the diaphragm and intercostal, abdominal, and pelvic floor muscles. The abdomen usually consciously rises and falls with a sensation of expansion or stretching, rather than mild exertion solely in their chest and shoulders as would happen in chest breathing. Diaphragmatic breathing can substantially increase the efficiency of the lungs. Breaths taken during diaphragmatic breathing are slow and deep, taking longer to inhale and exhale and delivering a significantly larger amount of oxygen to the bloodstream.

Breathing patterns such as diaphragmatic breathing and chest breathing can be affected by certain medical conditions. For example, obesity usually has a detrimental effect on the respiratory system, and may cause changes in breathing pattern. There is a significant association between body mass index (BMI) and pulmonary function parameters. High BMI is also associated with other disorders including, for example, diabetes, heart disease (e.g., heart failure), and various complications of metabolic syndrome. However, the risk of such disorders may vary among high BMI patients. Patients with a higher distribution of fat above the waist (thus an apple-shaped body) have been shown to have a higher risk of heart disease and metabolic disorders than those with fat predominantly distributed below the waist like in thighs and buttocks (thus a pear-shaped body). In obesity patients with an apple-shaped body, fat deposition within the abdominal and/or the thoracic cavities restrict the downward movement of the diaphragm and the outward movement of the chest wall, thereby reducing the capacity to perform diaphragmatic breathing.

Disturbance in breathing patterns (also known as disordered breathing) is associated with several pathological conditions, and may result in a variety of negative psychological, biochemical, neurological, and biomechanical influences and interferences. Patient with disordered breathing may present with hypopnea (shallow breathing), dyspnea (labored breathing), hyperpnea (deep breathing), tachypnea (rapid breathing), or a combinations of multiple breathing disorders thereof. For example, Cheyne-Stokes respiration (CSR) is a type of disordered breathing frequently observed in patients with congestive heart failure (CHF). CSR is associated with an increased risk of accelerated CHF progression is associated with rhythmic increases and decreases in tidal volume caused by alternating periods of hyperpnea followed by apnea or hypopnea.

Ambulatory medical devices (AMDs) have been used to monitor patients with chronic disease. Some AMDs include sensors to sense physiological signals from the patient. Some AMDs can provide diagnostic features. As a person's breathing patterns are generally associated with certain medical conditions, ambulatory monitoring of respiration pattern can help identify patients at an elevated risk of respiratory or cardiac disease, or various metabolic disorders. Some AMDs can deliver therapy such as electrical stimulations to target tissues or organs, such as to restore or improve cardiac performance.

### SUMMARY

Various types of disordered breathing may be associated with CHF. Rapid-shallow breathing (RSB) is a typical pattern associated with dyspnea caused by heart or lung disorders, strenuous activity, high anxiety or stress. RSB is different from tachypnea (rapid breathing) and hyperpnea (deep breathing). Tachypnea and hyperpnea can occur with hyperventilation, or over breathing beyond what is required to maintain arterial blood gases within normal limits, whereas hyperpnea may be an appropriate increase in breathing such as with exercise. RSB can be associated with symptoms of shortness of breath, or dyspnea. CHF patients frequently present with dyspnea with exertion, orthopnea (a sensation of breathlessness in a recumbent position), or paroxysmal nocturnal dyspnea (a sensation of shortness of breath that awakens the patient). Dyspnea may occur initially upon exertion, but in advanced CHF it may occur at rest, or when lying down. In diastolic heart failure, increased pressure can build up in the heart during the period of relaxation, or diastole. For example, the incidence of diaphragmatic breathing (or the lack thereof) may be indicative of a patient's risk of heart disease or other metabolic disorders due to visceral fat deposition in the chest wall and/or abdomen.

Implantable medical devices (IMDs) can monitor respiration and detect cardiopulmonary events, such as events leading to WHF. An IMD may provide ambulatory respiration monitoring, which is particularly desirable for patients at risk of cardiopulmonary events. These IMDs may include or be coupled to sensors or electrodes to sense a physiological signal, from which respiration may be sensed. However, ambulatory respiration monitoring may face some challenges. For example, some IMDs can sense respiration using impedance measurements via electrodes included in or otherwise coupled to the IMD. An IMD, such as an implantable cardiac monitor (ICM), may have a small size and slim profile. The sensed impedance signal may have weak signal strength and is prone to noises and various physiological or non-physiological interferences, such as motion artifacts. The impedance signal may also be affected by the device implant site and IMD orientation at the implant site. For these reasons, the present inventors have recognized that there remains a demand for techniques to more reliably recognize a breathing pattern, stratify a shallow breathing risk, detecting disordered breathing and other cardiac and respiratory events or conditions, such as WHF and metabolic disorders.

This document discusses, among other things, systems, devices and methods for recognizing different breathing patterns (e.g., diaphragmatic breathing and chest breathing) based on spatial analysis of chest or abdominal movement or acceleration. An exemplary medical-device system comprises a receiver circuit to receive respiration information of a patient, and a breath analyzer circuit to determine two or more spatial respiration components from the respiration information. The spatial respiration components include accelerations of chest wall or abdominal movement along respective anatomical axes of the patient, including a first respiration component along an anterior-posterior axis and one or more second respiration components along a superior-inferior axis or a left-right axis. The breath analyzer circuit can classify a breathing pattern as one of either chest breathing or diaphragmatic breathing based on the spatial respiration components. The classified breathing pattern can be used for detecting a physiological event such as worsening heart failure (WHF), or for assessing the patient's risk of metabolic disorders.

Example 1 is a medical-device system, comprising: a receiver circuit configured to receive respiration information of a patient; and a breath analyzer circuit configured to: determine, from the received respiration information, two or more spatial respiration components along respective anatomical axes of the patient, including a first respiration component along an anterior-posterior (AP) axis and one or more second respiration components along a superior-inferior (SI) axis or a left-right (LR) axis; and classify a breathing pattern as one of either chest breathing or diaphragmatic breathing using the determined two or more spatial respiration components.

In Example 2, the subject matter of Example 1 optionally includes at least one accelerometer sensor configured to sense multiple directional acceleration signals of chest wall or abdominal movement correlated to respiration, the multiple directional acceleration signals including an anterior-posterior acceleration (XL-AP) signal, a superior-inferior acceleration (XL-SI) signal, and a left-right acceleration (XL-LR) signal, wherein the breath analyzer circuit is configured to: determine the two or more spatial respiration components using respective signal metrics of the sensed multiple directional acceleration signals; and classify the breathing pattern as the diaphragmatic breathing if the signal metric of the XL-AP signal is greater than the signal metric of the XL-SI signal and the signal metric of the XL-LR signal, and to classify the breathing pattern as the chest breathing if the signal metric of the XL-AP signal is no greater than any of the signal metric of the XL-SI signal or the signal metric of the XL-LR signal.

In Example 3, the subject matter of Example 2 optionally includes, wherein to classify the breathing pattern, the breath analyzer circuit is configured to: calculate a breathing pattern score representing a relative strength of the signal metric of the XL-AP signal with respect to one or more of the signal metric of the XL-SI signal or the signal metric of the XL-LR signal; and classify the breathing pattern as the diaphragmatic breathing if the breathing pattern score exceeds a threshold, or as the chest breathing if the breathing pattern score is below the threshold.

In Example 4, the subject matter of any one or more of Examples 2-3 optionally includes the at least one accelerometer sensor that can include a plurality of single-axis accelerometers configured to sense, respectively, the multiple directional acceleration signals of chest wall or abdominal movement.

In Example 5, the subject matter of any one or more of Examples 2-4 optionally includes the at least one accelerometer sensor that can include a multi-axis accelerometer.

In Example 6, the subject matter of any one or more of Examples 2-5 optionally includes the breath analyzer circuit that can be configured to filter the sensed multiple directional acceleration signals to a respiration frequency range, and to determine the two or more spatial respiration components using the filtered multiple directional acceleration signals.

In Example 7, the subject matter of any one or more of Examples 2-6 optionally includes an ambulatory medical device (AMD) configured for abdomen or chest placement, wherein the at least one accelerometer sensor is included in the AMD and configured to sense the multiple directional acceleration signals along directions with respect to an orientation of the AMD, wherein the breath analyzer circuit is configured to determine a spatial relationship between the orientation of the AMD and the anatomical axes of the patient, and to calibrate the multiple directional acceleration signals using the determined spatial relationship.

In Example 8, the subject matter of any one or more of Examples 2-7 optionally includes the respective signal metrics of the sensed multiple directional acceleration signals that can include respective signal amplitudes or signal power within a respiration cycle.

In Example 9, the subject matter of any one or more of Examples 1-8 optionally includes the receiver circuit that can be configured to receive posture information of the patient when the respiration information is sensed, wherein the breath analyzer circuit is configured to determine the two or more spatial respiration components further using the posture information.

In Example 10, the subject matter of any one or more of Examples 1-9 optionally includes the receiver circuit that can be configured to receive physical activity information of the patient when the respiration information is sensed, wherein the breath analyzer circuit is configured to determine the two or more spatial respiration components further using the physical activity information.

In Example 11, the subject matter of any one or more of Examples 1-10 optionally includes the breath analyzer circuit that can be configured to: generate a trend for each of the two or more spatial respiration components over multiple respiration cycles; and classify the breathing pattern based at least in part on a comparison of generated trends of the two or more spatial respiration components.

In Example 12, the subject matter of any one or more of Examples 1-11 optionally includes a physiological event detector configured to detect a worsening heart failure event based at least in part on the classified breathing pattern.

In Example 13, the subject matter of any one or more of Examples 1-12 optionally includes a physiological event detector configured to determine a risk of metabolic disorder based at least in part on the classified breathing pattern.

In Example 14, the subject matter of Example 13 optionally includes the physiological event detector that can be configured to determine the risk of metabolic disorder further using a body mass index of the patient.

In Example 15, the subject matter of any one or more of Examples 1-14 optionally includes a physiological event detector, wherein the breath analyzer circuit is configured to, in response to a physical or emotional trigger event, detect a change or a rate of change in breathing pattern from the chest breathing to the diaphragmatic breathing or from the diaphragmatic breathing to the chest breathing, wherein the physiological event detector is configured to detect a physiological event or condition of the patient based at least in part on the detected change or the detected rate of change in breathing pattern.

Example 16 is a method of monitoring respiration using a medical-device system, the method comprising: receiving respiration information sensed from a patient; determining, from the received respiration information, two or more spatial respiration components along respective anatomical axes of the patient, including a first respiration component along an anterior-posterior (AP) axis and one or more second respiration components along a superior-inferior (SI) axis or a left-right (LR) axis; and classifying a breathing pattern as one of either chest breathing or diaphragmatic breathing using the determined two or more spatial respiration components.

In Example 17, the subject matter of Example 16 optionally includes the received respiration information that can include multiple directional acceleration signals of chest wall or abdominal movement correlated to respiration, the multiple directional acceleration signals including an anterior-posterior acceleration (XL-AP) signal, a superior-inferior acceleration (XL-SI) signal, and a left-right acceleration (XL-LR) signal, wherein determining the two or more spatial respiration components includes determining respective signal metrics of the multiple directional acceleration signals, wherein classifying the breathing pattern incudes classifying the diaphragmatic breathing if the signal metric of the XL-AP signal is greater than the signal metric of the XL-SI signal and the signal metric of the XL-LR signal, and classifying the chest breathing if the signal metric of the XL-AP signal is no greater than any of the signal metric of the XL-SI signal or the signal metric of the XL-LR signal.

In Example 18, the subject matter of Example 17 optionally includes classifying the breathing pattern that can include: calculating a breathing pattern score representing a relative strength of the signal metric of the XL-AP signal with respect to one or more of the signal metric of the XL-SI signal or the signal metric of the XL-LR signal; and classifying the breathing pattern as the diaphragmatic breathing if the breathing pattern score exceeds a threshold, or as the chest breathing if the breathing pattern score is below the threshold.

In Example 19, the subject matter of any one or more of Examples 17-18 optionally includes: sensing the multiple directional acceleration signals using at least one accelerometer sensor included in an ambulatory medical device (AMD) configured for abdomen or chest placement, the multiple directional acceleration signals being sensed along respective directions with respect to an orientation of the AMD; determining a spatial relationship between the orientation of the AMD and the anatomical axes of the patient; and calibrating the multiple directional acceleration signals using the determined spatial relationship.

In Example 20, the subject matter of any one or more of Examples 17-19 optionally includes the respective signal metrics of the multiple directional acceleration signals that can include respective signal amplitudes or signal power within a respiration cycle.

In Example 21, the subject matter of any one or more of Examples 16-20 optionally includes detecting a worsening heart failure event or a risk of metabolic disorder based at least in part on the classified breathing pattern.

In Example 22, the subject matter of any one or more of Examples 16-21 optionally includes, in response to a physical or emotional trigger event, detecting a change or a rate of change in breathing pattern from the chest breathing to the diaphragmatic breathing or from the diaphragmatic breathing to the chest breathing; and detecting a physiological event or condition of the patient based at least in part on the detected change or the detected rate of change in breathing pattern.

Various embodiments described herein can improve the medical technology of device-based respiration monitoring and disordered breathing detection. Breathing disturbances may be associated with a disease condition, such as WHF, sleep apnea, or other various cardiac, pulmonary, neurological, or psychological disorders. Monitoring respiratory disturbances may provide useful clinical diagnostic information, or trigger other types of patient monitoring or delivery of desired therapies. Compared to impedance-based respiration monitoring, the acceleration-based respiration monitoring and breathing pattern classification as described in this document in accordance with various embodiments are less complicated, and particularly more suitable for use in miniature implantable devices such as an ICM. Additionally, the acceleration-based breathing pattern classification may help improve the accuracy and efficiency of detecting and characterizing breathing disorders, stratifying shallow breathing risk, and detecting a respiratory or cardiac event. Accordingly, therapies may be timely provided or adjusted (e.g., ambulatory therapy such as cardiac pacing), hospitalization may be reduced, and healthcare costs associated with patient management may be reduced.

This Summary is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates generally an example of a patient management system and portions of an environment in which the system may operate.

FIG. 2 illustrates generally an example of a medical-device system for analyzing and classify breathing patterns and detecting a physiological event or condition of a subject.

FIG. 3 illustrates a coordinate system that describes a three-dimensional (3D) anatomical position of a subject and anatomical planes in such coordinate system.

FIGS. 4A-4B illustrate generally examples of directional acceleration signals of thoracic or abdominal movement during respiration.

FIG. 5 illustrates generally an example of a method of analyzing and classifying breathing patterns and detecting a physiological event or condition based on the breathing pattern classification.

FIG. 6 illustrates generally a block diagram of an example machine upon which any one or more of the techniques discussed herein may perform.

### DETAILED DESCRIPTION

Disclosed herein are systems, devices, and methods for monitoring and classifying breathing patterns (such as diaphragmatic breathing and chest breathing) based on spatial analysis of chest wall or abdominal movement or acceleration. An exemplary medical-device system comprises a receiver circuit to receive respiration information of a patient, and a breath analyzer circuit to determine from the respiration information two or more spatial respiration components, such as directional accelerations of thoracic or abdominal movement, along respective natural anatomical axes. The breath analyzer circuit can classify a breathing pattern as one of either chest breathing or diaphragmatic breathing using the determined spatial respiration components. The classified breathing pattern can be used for detecting for detecting a physiological event such as worsening heart failure or for assessing the patient's risk of having metabolic disorders.

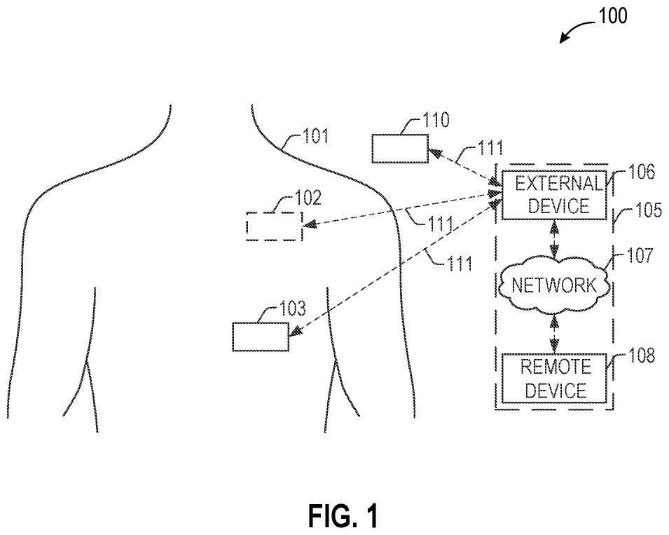

FIG. 1 illustrates generally an example patient management system 100 and portions of an environment in which the patient management system 100 may operate. The patient management system 100 can perform a range of activities, including remote patient monitoring and diagnosis of a disease condition. Such activities can be performed proximal to a patient 101, such as in a patient home or office, through a centralized server, such as in a hospital, clinic, or physician office, or through a remote workstation, such as a secure wireless mobile computing device.

The patient management system 100 may include one or more ambulatory medical devices, an external system 105, and a communication link 111 providing for communication between the one or more ambulatory medical devices and the external system 105. The one or more ambulatory medical devices may include an implantable medical device (IMD) 102, a wearable medical device (WMD) 103, or one or more other implantable, leadless, subcutaneous, external, wearable, or ambulatory medical devices configured to monitor, sense, or detect information from, determine physiological information about, or provide one or more therapies to treat various conditions of the patient 101, such as one or more cardiac or non-cardiac conditions (e.g., disordered breathing, metabolic disorders, etc.).

In an example, the IMD 102 may include one or more traditional cardiac rhythm management devices implanted in a chest of a patient, having a lead system including one or more transvenous, subcutaneous, or non-invasive leads or catheters to position one or more electrodes or other sensors (e.g., a respiration sensor, a heart sound sensor) in, on, or about a heart or one or more other position in a thorax, abdomen, or neck of the patient 101. In another example, the IMD 102 may include a monitor implanted, for example, subcutaneously in the chest of patient 101, the IMD 102 including a housing containing circuitry and, in certain examples, one or more sensors, such as a temperature sensor, etc.

The IMD 102 may include an assessment circuit configured to detect or determine specific physiological information of the patient 101, or to determine one or more conditions or provide information or an alert to a user, such as the patient 101 (e.g., a patient), a clinician, or one or more other caregivers or processes. In an example, the IMD 102 can be an implantable cardiac monitor (ICM) configured to collected cardiac information, optionally along with other physiological information, from the patient. The IMD 102 can alternatively or additionally be configured as a therapeutic device configured to treat one or more medical conditions of the patient 101. The therapy can be delivered to the patient 101 via the lead system and associated electrodes or using one or more other delivery mechanisms. The therapy may include delivery of one or more drugs to the patient 101, such as using the IMD 102 or one or more of the other ambulatory medical devices, etc. In some examples, therapy may include cardiac resynchronization therapy for rectifying dyssynchrony and improving cardiac function in heart failure patients. In other examples, the IMD 102 may include a drug delivery system, such as a drug infusion pump to deliver drugs to the patient for managing arrhythmias or complications from arrhythmias, hypertension, or one or more other physiological conditions. In other examples, the IMD 102 may include one or more electrodes configured to stimulate the nervous system of the patient or to provide stimulation to the muscles of the patient airway, etc.

The WMD 103 may include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

In an example, the IMD 102 or the WMD 103 may include or be coupled to an implantable or wearable sensor to sense respiration information. In an example, the respiration information may include chest wall or abdominal movement or acceleration that can be sensed using one more implantable or wearable accelerometers. The IMD 102 or the WMD 103 may analyze the respiration information and determine spatial respiration components, such as directional accelerations of thoracic or abdominal movement along respective natural anatomical axes, including a first respiration component along an anterior-posterior (AP) axis and one or more second respiration components along a superior-inferior (SI) axis or a left-right (LR) axis. The IMD 102 or the WMD 103 may classify a breathing pattern as one of either chest breathing or diaphragmatic breathing based on the spatial respiration components. The IMD 102 or the WMD 103 may further detect a physiological event such as a cardiac or respiratory event, or to assess the patient's risk of a medical condition such as a risk of metabolic syndrome, based at least on the classified breathing pattern.

The external system 105 may include a dedicated hardware/software system, such as a programmer, a remote server-based patient management system, or alternatively a system defined predominantly by software running on a standard personal computer. The external system 105 can manage the patient 101 through the IMD 102 or one or more other ambulatory medical devices connected to the external system 105 via a communication link 111. In other examples, the IMD 102 can be connected to the WMD 103, or the WMD 103 can be connected to the external system 105, via the communication link 111. This may include, for example, programming the IMD 102 to perform one or more of acquiring physiological data, performing at least one self-diagnostic test (such as for a device operational status), analyzing the physiological data, or optionally delivering or adjusting a therapy for the patient 101. In some examples, at least a portion of functionalities such as analyzing and classifying the breathing pattern and/or detecting the physiological event may be implemented in and executed by the external system 105. Additionally, the external system 105 can send information to, or receive information from, the IMD 102 or the WMD 103 via the communication link 111. Examples of the information may include real-time or stored physiological data from the patient 101, diagnostic data, such as detection of patient hydration status, hospitalizations, responses to therapies delivered to the patient 101, or device operational status of the IMD 102 or the WMD 103 (e.g., battery status, lead impedance, etc.). The communication link 111 can be an inductive telemetry link, a capacitive telemetry link, or a radio-frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 105 may include an external device 106 in proximity of the one or more ambulatory medical devices, and a remote device 108 in a location relatively distant from the one or more ambulatory medical devices, in communication with the external device 106 via a communication network 107. Examples of the external device 106 may include a medical device programmer. The remote device 108 can be configured to evaluate collected patient or patient information and provide alert notifications, among other possible functions. In an example, the remote device 108 may include a centralized server acting as a central hub for collected data storage and analysis. The server can be configured as a uni-, multi-, or distributed computing and processing system. The remote device 108 can receive data from multiple patients. The data can be collected by the one or more ambulatory medical devices, among other data acquisition sensors or devices associated with the patient 101. The server may include a memory device to store the data in a patient database. The server may include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications may include a Web page update, phone or pager call, E-mail, SMS, text or “Instant” message, as well as a message to the patient and a simultaneous direct notification to emergency services and to the clinician. Other alert notifications are possible. The server may include an alert prioritizer circuit configured to prioritize the alert notifications. For example, an alert of a detected physiological event can be prioritized using a similarity metric between the physiological data associated with the detected physiological event to physiological data associated with the historical alerts.

The remote device 108 may additionally include one or more locally configured clients or remote clients securely connected over the communication network 107 to the server. Examples of the clients may include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 108, including the server and the interconnected clients, may also execute a follow-up scheme by sending follow-up requests to the one or more ambulatory medical devices, or by sending a message or other communication to the patient 101 (e.g., the patient), clinician or authorized third party as a compliance notification.

The communication network 107 can provide wired or wireless interconnectivity. In an example, the communication network 107 can be based on the Transmission Control Protocol/Internet Protocol (TCP/IP) network communication specification, although other types or combinations of networking implementations are possible. Similarly, other network topologies and arrangements are possible.

One or more of the external device 106 or the remote device 108 can output the detected physiological events to a system user, such as the patient or a clinician, or to a process including, for example, an instance of a computer program executable in a microprocessor. In an example, the process may include an automated generation of recommendations for anti-arrhythmic therapy, or a recommendation for further diagnostic test or treatment. In an example, the external device 106 or the remote device 108 may include a respective display unit for displaying the physiological or functional signals, or alerts, alarms, emergency calls, or other forms of warnings to signal the detection of arrhythmias. In some examples, the external system 105 may include an external data processor configured to analyze the physiological or functional signals received by the one or more ambulatory medical devices, and to confirm or reject the detection of arrhythmias. Computationally intensive algorithms, such as machine-learning algorithms, can be implemented in the external data processor to process the data retrospectively to detect cardia arrhythmias.

Portions of the one or more ambulatory medical devices or the external system 105 can be implemented using hardware, software, firmware, or combinations thereof. Portions of the one or more ambulatory medical devices or the external system 105 can be implemented using an application-specific circuit that can be constructed or configured to perform one or more functions or can be implemented using a general-purpose circuit that can be programmed or otherwise configured to perform one or more functions. Such a general-purpose circuit may include a microprocessor or a portion thereof, a microcontroller or a portion thereof, or a programmable logic circuit, a memory circuit, a network interface, and various components for interconnecting these components. For example, a “comparator” may include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” may include electronic circuits configured to receive information and provide an electronic output representative of such received information.

The therapy device 110 can be configured to send information to or receive information from one or more of the ambulatory medical devices or the external system 105 using the communication link 111. In an example, the one or more ambulatory medical devices, the external device 106, or the remote device 108 can be configured to control one or more parameters of the therapy device 110. The external system 105 can allow for programming the one or more ambulatory medical devices and can receives information about one or more signals acquired by the one or more ambulatory medical devices, such as can be received via a communication link 111. The external system 105 may include a local external implantable medical device programmer. The external system 105 may include a remote patient management system that can monitor patient status or adjust one or more therapies such as from a remote location.

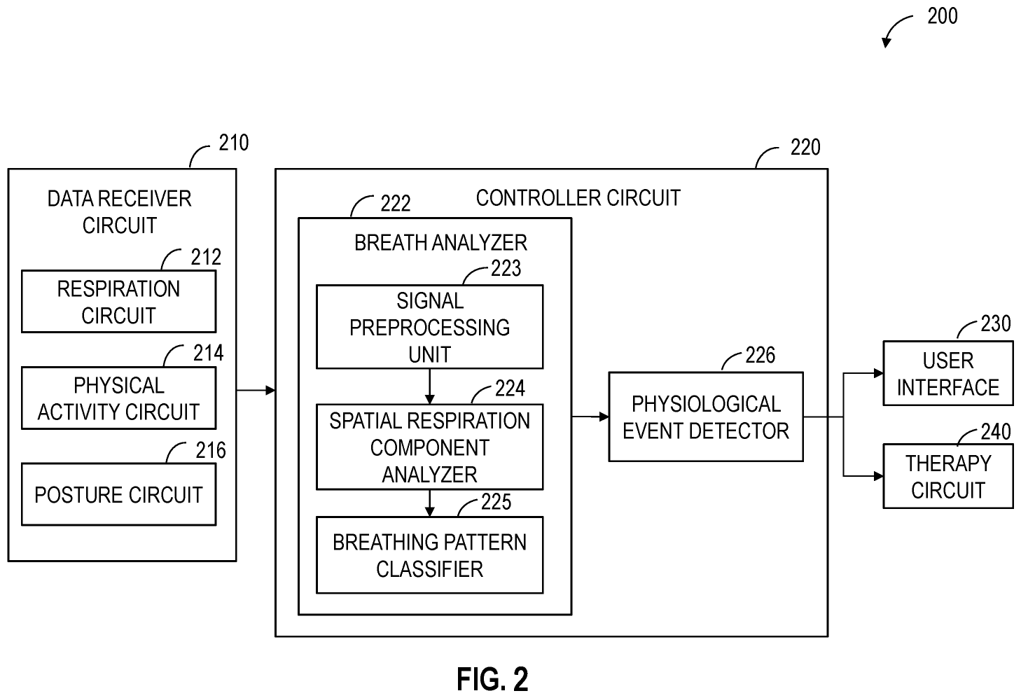

FIG. 2 illustrates generally an example of a medical-device system 200 for analyzing and classify breathing patterns and detecting a physiological event or condition of a subject. At least a portion of the system 200 may be implemented in the IMD 102, the WMD 103, or the external system 105 such as one or more of the external device 106 or the remote device 108.

The system 200 may include a data receiver circuit 210, a controller circuit 220, a user interface 230, and a therapy circuit 240. The data receiver circuit 210 may receive physiological information from a patient. In an example, the data receiver circuit 210 may sense a physiological signal from a patient via a sensor, such as an implantable, wearable, or otherwise ambulatory sensor or electrodes associated with the patient. The sensor may be incorporated into, or otherwise associated with an ambulatory device such as the IMD 102 or the WMD 103. In some examples, the physiological signals sensed from a patient may be stored in a storage device, such as an electronic medical record system. The data receiver circuit 210 may receive the physiological signal from the storage device, such as in response to a user command or a triggering event.

By way of example and not limitation and as illustrated in FIG. 2, the data receiver circuit 210 may include a respiration circuit 212, and optionally one or more of a physical activity circuit 214 or a posture circuit 216. The respiration circuit 212 can may be coupled to one or more implantable, wearable, holdable, or other ambulatory respiratory sensors to sense respiration information. The respiration circuit 212 may each include sub-circuits to digitize, filter, or perform other signal conditioning operations on the sensed respiration information. In an example, the respiration circuit 212 may be included in one medical device, such as the IMD 102 or the WMD 103.

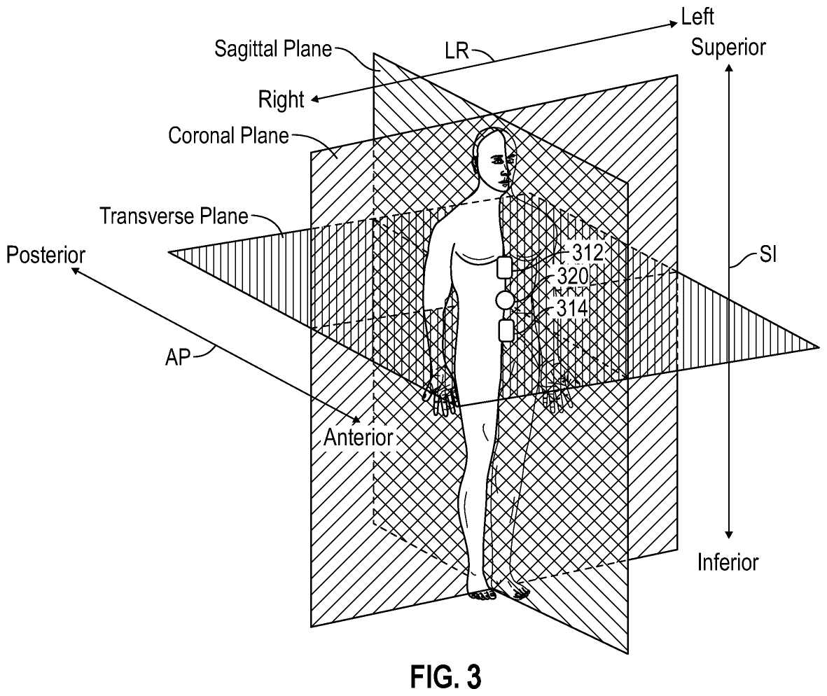

In an example, the respiration circuit 212 may be coupled to at least one accelerometer attached to or implanted in the patient to sense movement or acceleration of a body part of the patient that are correlated to respiration. The accelerometer may take the form of, for example, a piezoelectric accelerometer such as one employing piezoelectric crystals, a capacitive accelerometer, a strain gauge accelerometer, or a Hall-effect accelerometer that senses a change in magnetic field, or other micro-machined micro-electromechanical systems (MEMS) accelerometers. In an example, the accelerometer can be a single-axis accelerometer configured to sense movement or acceleration in a specific direction. In another example, the accelerometer can be a multi-axis accelerometer configured to simultaneously sense movements or accelerations in multiple directions. In an example, the directions of the movement or acceleration can be aligned with the patient's anatomical axes, such as an anterior-posterior (AP) axis, an superior-inferior (SI) axis, and a left-right (LR) axis. Referring to FIG. 3, the diagram therein illustrates a coordinate system used for describing a three-dimensional (3D) anatomical position of a subject. The coordinate system comprises an anterior-posterior (AP) axis going from front to back, a left-right (LR) axis going from left to right, and a superior-inferior (SI) axis (also known as craniocaudal axis) going from up to down. The anatomical axes define three anatomical planes in the coordinate system, including a sagittal plane formed by the SI and AP axes, a coronal plane formed by the SI and LR axes, and a transvers plane formed by the LR and AP axes. In an example, a plurality of single-axis accelerometers can each be positioned at respective different body locations, such as on the upper torso, to sense local body movement and acceleration therein. For example, a first accelerometer 312 may be positioned in a front chest location to sense chest wall motion or acceleration representing chest expansion and contraction during respiration, predominantly along the SI axis. A second accelerometer 314 may be positioned in an abdominal location to sense abdominal motion or acceleration representing diaphragm contraction (tightening) and relaxation during respiration, predominantly along the AP axis. In another example, a multi-axis accelerometer 320 may be positioned at a specific body location, such as approximately the middle of the chest near sternum or diaphragm, to sense multi-axis accelerations along respective directions. The multi-axis accelerations may include, for example, a first acceleration component along the AP axis and one or more second acceleration components along the SI axis or the LR axis.

In addition or alternative to the accelerometers, body movement associated with respiration, such as chest wall motion or abdominal motion during respiration, may be sensed using a gyrometer or gyroscope, a pressure sensor, a magnetometer (e.g., a compass), an inclinometer, a sensing fabric, a force sensor, a strain gauge, an electromyography (EMG) sensor, among other sensor modalities. The gyrometer or the gyroscope can be included in an ambulatory device, such as the IMD 102 or the WMD 103, to sense the orientation or a change in orientation of the device indicative of or correlated to respiration-induced chest or abdominal movement along specific direction(s). The gyrometer or the gyroscope can be a single-axis sensor; or alternatively a multi-axis sensor. Similar to the multiple single-axis accelerometers or a multi-axis accelerometer as described above, multiple single-axis gyroscopes or one multi-axis gyroscope may be used to detect two or more spatial respiration components along respective anatomical axes including, for example, a first respiration component along the axis and one or more second respiration components along the SI axis or the LR axis.

In some examples, the respiration circuit 212 may sense impedance via electrodes attached to or implanted in the patient. An example of the sensed impedance includes a thoracic impedance representing an electrical property of the chest and varies during inspiration expiration phases, such that the impedance increases during inspiration and decreases during expiration. Electrical current may be injected into a body part (e.g., the chest) between two stimulation electrodes to establish an electric field that covers at least a portion of the chest, and voltage drop may be measured between a pair of sensing electrodes. The impedance may be determined using Ohm's law. The impedance-sensing electrodes may be associated with an implantable lead coupled to an implantable medical device. By way of example and not limitation, impedance may be measured between an electrode on a right ventricular and the can housing of the implantable device implanted at a pectoral region, between an electrode on a left ventricle and the can housing of the implantable device, or between a right atrium electrode and the can housing of the implantable device. Alternatively, impedance-sensing electrodes may be included within or on an ambulatory physiological monitor, such as an implantable cardiac monitor. The impedance vector, such as defined by a pair of voltage-sensing electrodes, may be constrained by the size and shape of the implantable cardiac monitor. In an example, the implantable cardiac monitor has a small size and slim profile. Accordingly, the impedance vector may be a small vector characterized by relative short spacing between the voltage-sensing electrodes and covers a small portion of the chest. The small vector impedance may be sensitive to implant site location and orientation of the implantable cardiac monitor. In some examples, impedance may be measured using non-invasive surface electrodes removably attached to a patient chest.

In some examples, the respiration circuit 212 may be coupled to a flowmeter that directly senses airflow in the respiratory system or volume change in the lungs. In another example, respiration may be sensed using one or more of a strain sensor configured to sense changes in chest muscle tension corresponding to respiration cycles, an accelerometer to measure acceleration associated with displacement or movement of chest walls corresponding to respiration, or an acoustic sensor to sense cardiac acoustic signal that is modulated by respiration. In yet another example, respiration may be extracted from a cardiac electrical signal modulated by respiratory signal, such as an ECG signal. During inspiration, the diaphragm shift downwards away from the apex of the heart. The increased filling of the lungs further stretches the apex of the heart towards the abdomen. During expiration, the lung volume reduces, and the diaphragm elevates upwards toward the heart, which compresses the apex of the heart towards the breast. As a result, the angle of the electric cardiac vector that gives rise to the ECG signal changes during inspiration and respiratory phases, which leads to cyclic variation in R-wave amplitude on the ECG signal. The respiratory signal can be obtained from the R-wave amplitude signal using demodulation method, such as by filtering an R-wave amplitude trend through a low-pass filter or a bandpass filter.

The data receiver circuit 210 may additionally include one or more of a physical activity circuit 214 or a posture circuit 216. The physical activity circuit 214 may be coupled to an accelerometer configured to sense a physical activity signal. The accelerometer for sensing physical activity may be different from the accelerometer for detecting respiration-induced chest wall or abdominal movement or acceleration. In an example, an accelerometer may be included in a separate device other than the IMD 102 or the WMD 103, such as a smart watch or other wearable devices, to sense physical activity information. The data receiver circuit 210 can be communicate with the separate device and received therefrom the physical activity information. In another example, the same accelerometer used for sensing the respiration-induced chest wall or abdominal movement or acceleration may also be used for sensing physical activity. Acceleration signals sensed by the at least one accelerometer may be filtered to obtain a first respiration component (XL₁) within a respiration frequency band, and a second signal (XL₂) outside the respiration frequency band. XL₁ may be used for analyzing and classifying a breathing pattern (e.g., by the breath analyzer circuit 222 as described further below). XL₂ may be used for detecting a physical activity level. The posture circuit 216 may be coupled to a posture sensor, which can take the form of a tilt switch or a single- or multi-axis accelerometer associated with the patient. The posture sensor may be disposed external to the body or implanted inside the body. Posture may be represented by, for example, a tilt angle.

In some examples, the posture or physical activity information may be derived from thoracic impedance information. The respiration sensor may include a flowmeter that directly senses airflow in the respiratory system or volume change in the lung, a strain sensor configured to sense changes in chest muscle tension corresponding to respiration cycles, an accelerometer to measure acceleration associated with displacement or movement of chest walls corresponding to respiration, or an impedance sensor to sense thoracic impedance that is modulated by respiration.

The controller circuit 220 can analyze respiration and classify a breathing pattern, and detect a physiological event or condition based at least in part on the classified breathing pattern. The controller circuit 220 can be implemented as a part of a microprocessor circuit, which may be a dedicated processor such as a digital signal processor, application specific integrated circuit (ASIC), microprocessor, or other type of processor for processing information including physical activity information. Alternatively, the microprocessor circuit may be a general-purpose processor that may receive and execute a set of instructions of performing the functions, methods, or techniques described herein.

The controller circuit 220 may include circuit sets comprising one or more other circuits or sub-circuits, such as a breath analyzer circuit 222 and a physiological event detector 226. These circuits may, alone or in combination, perform the functions, methods, or techniques described herein. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

The breath analyzer circuit 222 may include a signal preprocessing unit 223, a spatial respiration component analyzer 224, and a breathing pattern classifier 225. The signal preprocessing unit 223 may include a filter or a filter bank to remove or attenuate one or more of low-frequency signal baseline drift, high frequency noise, or other unwanted frequency contents from the received respiration information, such as the acceleration signals correlated to respiration. In an example, the signal preprocessing unit 223 may detect a respiration cycle from the respiration information received from the respiration circuit 212, and select a portion of the received respiration information within the detected respiration cycle for subsequent breath analysis. In an example, the signal preprocessing unit 223 may select a portion of the received respiration information based on the patient's physical activity level detected or received by the physical activity circuit 214, such as the portion of the respiration information sensed when the physical activity level is below a threshold. In another example, the signal preprocessing unit 223 may select a portion of the received respiration information based on the patient's posture detected or received by the posture circuit 216, such as the portion of the respiration information sensed when the patient is in a supine position. In some examples, the signal preprocessing unit 223 may additionally or alternatively select a portion of the received respiration information based on the patient's health status, heart rate, respiration rate, sleep state, or a time of a day (e.g., during the nighttime) when the patient would normally be involved in low or mild physical activity.

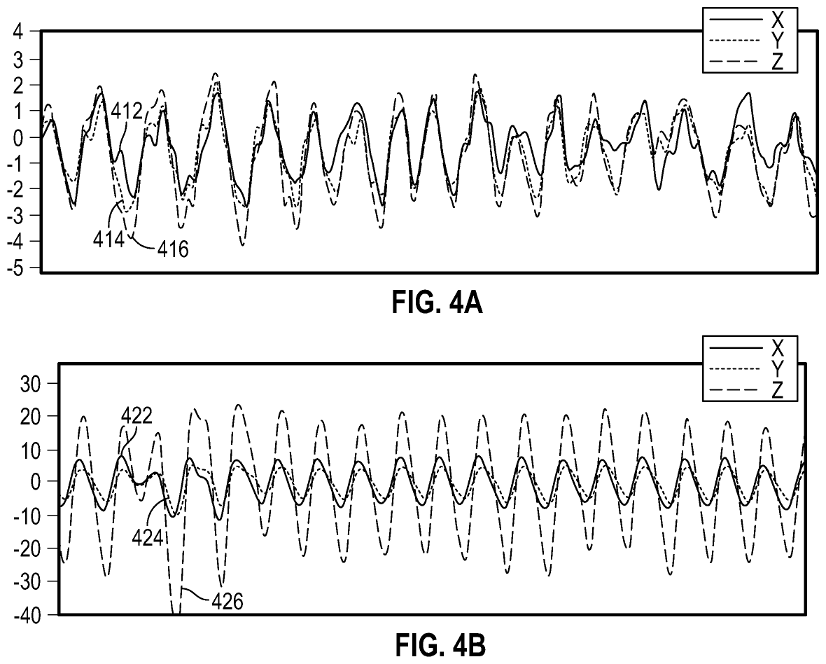

The spatial respiration component analyzer 224 can determine two or more spatial respiration components from the preprocessed respiration information. The two or more spatial respiration components can be determined along two or more anatomical axes of the patient, including a first respiration component along the AP axis and one or more second respiration components along the SI axis or the LR axis. In an example where at least one accelerometer sensor is used to sense the respiration information, the two or more spatial respiration components can be determined using multiple directional acceleration signals of thoracic or abdominal movement correlated to respiration, which may include an anterior-posterior acceleration (XL-AP) signal, a superior-inferior acceleration (XL-SI) signal, and a left-right acceleration (XL-LR) signal. The multiple directional acceleration signals can be respectively sensed using a plurality of single-axis accelerometers, such as accelerometers 312 and 314 as shown in FIG. 3. Alternatively, the multiple directional acceleration signals can be obtained by spatial separation of the acceleration signal sensed using a multi-axis accelerometer, such as the multi-axis accelerometer 320 as shown in FIG. 3. Examples of directional acceleration signals of thoracic or abdominal movement during respiration are described below with reference to FIGS. 4A and 4B.

The spatial respiration component analyzer 224 calculate a signal metric for each of the directional acceleration signals. By way of example and not limitation, the signal metric can include signal amplitude or signal power respectively measured or determined from the XL-AP signal, the XL-SI signal, and the XL-LR signal. As described above, the directional acceleration signals may be filtered into a respiration frequency range, and the signal metrics can be calculated using the respectively filtered directional acceleration signals.

In some examples, at least one accelerometer (e.g., one of the single-axis accelerometers, or the multi-axis accelerometer) can be included in an ambulatory device, such as the IMD 102 or the WMD 103. The ambulatory device can be configured for chest or abdominal placement. The at least one accelerometer sensor included therein can sense the multiple directional acceleration signals along directions in a coordinate system with respect to an orientation of the ambulatory device. Such coordinate system may not be substantially aligned with the anatomical coordinate system spanned by the anatomical axes (e.g., the AP axis, the SI axis, and the LR axis) as illustrated in FIG. 3. To determine the directional acceleration signals along the anatomical axes (i.e., the XL-AP signal, the XL-SI signal, and the XL-LR signal), the spatial respiration component analyzer 224 can determine a spatial relationship between the orientation of the ambulatory device and the anatomical axes of the patient, and calibrate the directional acceleration signals using such determined spatial relationship. The spatial relationship can be represented by, for example, a transformation matrix, also referred to as a rotation matrix, from the coordinate system with respect to the orientation of the ambulatory device to the coordinate system defined by the anatomical axes as illustrated in FIG. 3. Commonly assigned U.S. patent application Ser. No. 16/940,250 entitled “CALIBRATION OF IMPLANTABLE DEVICE ORIENTATION”, filed on Jul. 27, 2020, describes techniques for calibrating an orientation of an implantable device such that the devices axes are substantially aligned with reference axes such as the body axes of a patient, the disclosure of which is incorporated herein by reference in its entirety.

The breathing pattern classifier 225 can classify a breathing pattern as one of either chest breathing or diaphragmatic breathing using the two or more spatial respiration components determined by the spatial respiration component analyzer 224. In an example, the classification of breathing pattern can be based on a comparison of the signal metrics of the directional acceleration signals, such as signal amplitude or signal power of the XL-AP signal, the XL-SI signal, and the XL-LR signal. The breathing pattern can be classified as diaphragmatic breathing if the respiration component along the AP axis (e.g., the XL-AP signal) is greater than the respiration components along the SI axis and the LR axes (e.g., the XL-SI signal and the XL-LR signal). The breathing pattern can be classified as chest breathing if the respiration component along the AP axis (e.g., the XL-AP signal) is no greater than any of the respiration components along the SI axis or the LR axes (e.g., the XL-SI signal or the XL-LR signal).

In some examples, the breath pattern classifier 225 can calculate a breathing pattern score using the signal metric of the respiration component along the AP axis (e.g., the XL-AP signal) relative to one or more of the respective signal metrics of the respiration components along the SI axis or the LR axes (e.g., the XL-SI signal or the XL-LR signal). The breathing pattern score indicates a likelihood of the breathing pattern being diaphragmatic (or being chest breathing). By way of example and not limitation, the breathing pattern score can be computed as a ratio of the signal metric (e.g., a signal amplitude or signal power) of XL-AP to the signal metric of XL-SI, a ratio of the signal metric of XL-AP to the signal metric of XL-LR, a ratio of the signal metric of XL-AP to the sum of the signal metrics of XL-SI and XL-LR, or a ratio of the signal metric of XL-AP to the sum of the signal metrics of XL-SI, XL-LR, and XL-AP. The breathing pattern score can take a value between zero and one, where a larger score indicates a high likelihood of diaphragmatic breathing and a lower likelihood of chest breathing, and vice versa. The breath pattern classifier 225 can classify the breathing pattern as diaphragmatic breathing if the breathing pattern score exceeds a threshold, or as chest breathing if the breathing pattern score is below the threshold.

In some examples, the breath pattern classifier 225 can generate, for each of the two or more spatial respiration components, a respective signal metric trend over multiple respiration cycles or over a time period, such as a XL-AP trend, a XL-SI trend, and a XL-LR trend. The breath pattern classifier 225 can classify the breathing pattern based on a comparison of the signal metric trends. For example, an increasing XL-AP trend, optionally accompanied by a decreasing XL-SI trend, can be an indicator of diaphragmatic breathing; while an increasing XL-SI trend, optionally accompanied by a decreasing XL-AP trend, can be an indicator of chest breathing.

The physiological event detector 226 can detect a physiological event or condition based at least in part on the classified breathing pattern. Examples of the physiological events or conditions being detected may include cardiac or respiratory events, pulmonary edema, sleep apnea, COPD, asthma, pulmonary embolism, or breathing disturbance or disorders associated with other medical conditions such as diabetic ketoacidosis. In an example, the physiological event or condition being detected includes worsening heart failure (WHF). WHF can be associated with respiratory distress and disordered breathing, such as a substantial increase in rapid shallow breathing. The physiological event detector 226 may detect an amount of time spent in chest breathing during a specific monitoring or diagnosis time period. The physiological event detector 226 may detect an increase in rapid shallow breathing if the chest breathing time exceeds a time threshold, or exceeds a previously determined baseline chest breathing time by a difference threshold. The physiological event detector 226 can detect the WHF based at least in part on the detected increase in rapid shallow breathing.

In an example, the physiological event detector 226 can detect a risk of metabolic disorder based at least in part on the classification of the breathing pattern. Metabolic disorders may be associated with various health issues and chronic conditions such as obesity, diabetes, heart disease, among others. Obesity patients with a higher distribution of fat above the waist may have a higher risk of heart disease and metabolic disorders than those with fat predominantly distributed below the waist. Deposition of fat within the thoracic and abdominal cavities may reduce an individual's capacity to perform diaphragmatic breathing. In an example, the physiological event detector 226 may determine an amount of time spent in diaphragmatic breathing or an amount of time spent in chest breathing during a specific monitoring or diagnosis time period, and determine the metabolic disorder risk based on a decrease (or a rate of decrease) in diaphragmatic breathing time, or based on an increase (or a rate of increase) in chest breathing time. For example, if the diaphragmatic breathing time falls below a threshold, or if the chest breathing time exceeds a time threshold, then an elevated metabolic disorder risk can be determined. In some examples, to increase the risk stratification sensitivity or specificity, the physiological event detector 226 may determine the metabolic disorder risk further using the patient's body mass index (BMI), such that a higher BMI corresponds to a higher risk of metabolic disorder.

In some examples, the physiological event detector 226 can detect a change or a rate of change in breathing pattern, such as a change from chest breathing to diaphragmatic breathing, or from diaphragmatic breathing to chest breathing. The change or the rate of change in breathing pattern can be detected in response to a trigger event, such as physical exertion or emotional distress. The physiological event detector 226 can detect a physiological event or condition based at least in part on the detected change or the detected rate of change in breathing pattern in response to the trigger event. Breathing patterns such as chest breathing or diaphragmatic breathing can by dynamic and affected by a subject's physical or emotional state (e.g., stress levels). To capture such dynamics of breathing pattern, the physiological event detector 226 can track changes in breathing pattern before, during, and after a trigger event, such as physical or emotional exertion. If a rate of change from diaphragmatic breathing (deep breathing) to chest breathing (shallow breathing) in response to the trigger event exceeds a threshold, then the physiological event detector 226 can determine a higher risk of metabolic disorder and less tolerance to the trigger event. Conversely, if a rate of change from chest breathing (shallow breathing) to diaphragmatic breathing (deep breathing) following the termination of the trigger event exceeds a threshold (indicating quick “recovery” to diaphragmatic breathing), then a lower risk of metabolic disorder and a better tolerance to such trigger event can be determined.

The user interface 230 may include an input unit and an output unit. In an example, at least a portion of the user interface 230 may be implemented in the external system 105. The input unit may receive user input for programming the data receiver circuit 210 and the controller circuit 220, such as parameters for processing the respiration information (e.g., acceleration signals from one or more accelerometers), detecting spatial respiration components, classifying a breathing pattern, or detecting a physiological event or condition. The input unit may include a keyboard, on-screen keyboard, mouse, trackball, touchpad, touch-screen, or other pointing or navigating devices. The output unit may include a display for displaying the sensed heart sound signal, the representative heart sound segments, the spectral entropy time series, the heart sound metrics, information about the detected physiological events, and any intermediate measurements or computations, among others. The output unit may also present to a user, such as via a display unit, the therapy titration protocol and recommended therapy, including a change of parameters in the therapy provided by an implanted device, the prescription to get a device implanted, the initiation or change in a drug therapy, or other treatment options of a patient. The output unit may include a printer for printing hard copies of information that may be displayed on a display unit. The signals and information may be presented in a table, a chart, a diagram, or any other types of textual, tabular, or graphical presentation formats. The presentation of the output information may include audio or other media format. In an example, the output unit may generate alerts, alarms, emergency calls, or other forms of warnings to signal the system user about the detected medical events.

The therapy circuit 240 may be configured to deliver a therapy to the patient, such as in response to the detected physiological event. The therapy may be preventive or therapeutic in nature such as to modify, restore, or improve patient neural, cardiac, or respiratory functions. Examples of the therapy may include electrostimulation therapy delivered to the heart, a nerve tissue, other target tissues, a cardioversion therapy, a defibrillation therapy, or drug therapy including delivering drug to the patient. In some examples, the therapy circuit 240 may modify an existing therapy, such as adjust a stimulation parameter or drug dosage.

FIGS. 4A-4B illustrate generally examples of directional acceleration signals of thoracic or abdominal movement acquired from a patient during chest breathing (FIG. 4A) or diaphragmatic breathing (FIG. 4B). The directional acceleration signals can be measured simultaneously using a plurality of single-axis accelerometers positioned at respective different body locations on the upper torso, such as accelerometers 312 and 314 as shown in FIG. 3; or alternatively measured using a multi-axis accelerometer, such as the multi-axis accelerometer 320 as shown in FIG. 3. Three In particular, FIG. 4A illustrates directional acceleration signals along superior-inferior direction (XL-SI signal) 412, left-right acceleration (XL-LR signal) 414, and anterior-posterior direction (XL-AP signal) 416 during chest breathing. FIG. 4B illustrates XL-SI signal 422, XL-LR signal 424, and XL-AP signal 426 during diaphragmatic breathing. As illustrated in this example, the intensity (e.g., amplitude) of XL-AP signal 416 is no greater than the intensities of XL-SI signal 412 or XL-LR signal 414 during chest breathing. During diaphragmatic breathing, the intensity of XL-AP signal 426 increases significantly and exceeds the signal intensities of XL-SI signal 422 and XL-LR signal 424. A comparison of the signal intensity (or other signal metrics) of XL-AP signal and the intensities (or other signal metrics) of XL-SI and/or XL-LR signals can be used to distinguish diaphragmatic breathing from chest breathing, as described above with reference to FIG. 2.

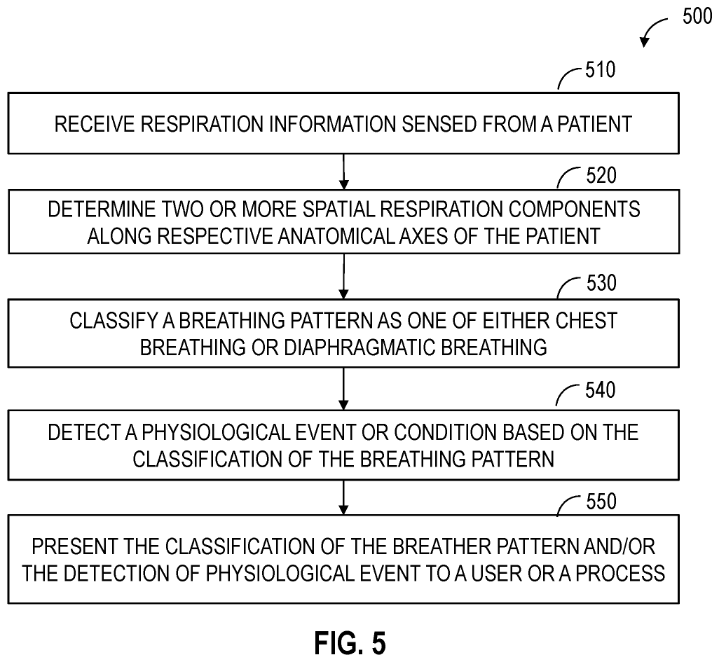

FIG. 5 illustrates generally an example of a method 500 for analyzing and classifying breathing patterns and detecting a physiological event or condition based on the breathing pattern classification. The method 500 may be implemented and executed in one or more ambulatory medical devices, such as the IMD 102 or the WMD 103, or the external system 105 such as one or more of the external device 106 or the remote device 108.

At 510, respiration information may be sensed from a subject, such as using the respiration circuit 212 coupled to one or more implantable, wearable, holdable, or other ambulatory respiratory sensors. In an example, the respiration information may be sensed using at least one accelerometer attached to or implanted in the patient to sense movement or acceleration of a body part of the patient correlated to respiration. In an example, the accelerometer can be a single-axis accelerometer configured to sense movement or acceleration in a specific direction. In another example, the accelerometer can be a multi-axis accelerometer configured to simultaneously sense movements or accelerations in multiple directions. The directions of the movement or acceleration can be aligned with the patient's anatomical axes, such as an anterior-posterior (AP) axis, an superior-inferior (SI) axis, and a left-right (LR) axis, as illustrated in FIG. 3, thus the corresponding directional acceleration signals including XL-SI signal along SI direction, XL-LR signal along the LR direction, and XL-AP signal along the AP direction.

The received respiration information, such as the acceleration signals correlated to respiration, can be preprocessed before being further analyzed such as for classifying a breath pattern. The preprocessing may include filtering the acceleration signals (e.g., the XL-A, XL-SI, and XL-LR signals) each to a specific respiration frequency range. The preprocessing may include selecting a portion of an acceleration signal for subsequent analysis, such as a portion within a respiration cycle. Additionally or alternatively, the portion of the acceleration signal may be selected based on a patient's physical activity level (e.g., during the time when the physical activity level is below a threshold level) or posture (e.g., during the time when the patient is in a supine position). The physical activity and posture may be sensed using respective sensor and/or detectors, such as the physical activity circuit 214 and the posture circuit 216, respectively. In some examples, the portion of the acceleration signal may be selected based on patient health status, heart rate, respiration rate, sleep state, or a time of a day (e.g., during the nighttime).

At 520, two or more spatial respiration components can be determined from the received respiration information. The along respective anatomical axes of the patient. The two or more spatial respiration components can be determined along two or more anatomical axes of the patient, including a first respiration component along the AP axis and one or more second respiration components along the SI axis or the LR axis. In an example, the two or more spatial respiration components can be determined using the multiple directional acceleration signals, such as the XL-AP signal, the XL-SI signal, and the XL-LR signal. A signal metric can be determined for each of the directional acceleration signals. Examples of the signal metric may include a signal amplitude or a signal power.

In some examples, the multiple directional acceleration signals can be sensed using at least one accelerometer sensor included in an ambulatory medical device (such as the IMD 102 or the WMD 103) that is configured for abdomen or chest placement. The sensed multiple directional acceleration signals may be along respective directions in a coordinate system with respect to an orientation of the AMD. Such coordinate system, however, may not be substantially aligned with the anatomical coordinate system as illustrated in FIG. 3. To determine the directional acceleration signals along the anatomical axes (i.e., the XL-AP signal, the XL-SI signal, and the XL-LR signal), a spatial relationship between the orientation of the ambulatory device and the patient's anatomical axes can be determined and used for calibrating the directional acceleration signals, as described above with reference to FIG. 2.

At 530, a breathing pattern can be classified as one of either chest breathing or diaphragmatic breathing using the determined two or more spatial respiration components. The classification of breathing pattern can be based on a comparison of the signal metrics of the directional acceleration signals, such as signal amplitude or signal power of the XL-AP signal, the XL-SI signal, and the XL-LR signal. The breathing pattern can be classified as diaphragmatic breathing if the respiration component along the AP axis (e.g., the XL-AP signal) is greater than the respiration components along the SI axis and the LR axes (e.g., the XL-SI signal and the XL-LR signal). The breathing pattern can be classified as chest breathing if the respiration component along the AP axis (e.g., the XL-AP signal) is no greater than any of the respiration components along the SI axis or the LR axes (e.g., the XL-SI signal or the XL-LR signal). In some examples, a breathing pattern score can be calculated using the signal metric of the XL-AP signal relative to one or more of the respective signal metrics of the XL-SI signal or the XL-LR signal. The breathing pattern score indicates a likelihood of the breathing pattern being diaphragmatic (or being chest breathing). By way of example and not limitation, the breathing pattern score can be computed as a ratio of the signal metric (e.g., a signal amplitude or power) of XL-AP to the signal metric of XL-SI, a ratio of the signal metric of XL-AP to the signal metric of XL-LR, a ratio of the signal metric of XL-AP to a sum of the signal metrics of XL-SI and XL-LR, or a ratio of the signal metric of XL-AP to a sum of the signal metrics of XL-SI, XL-LR, and XL-AP. The breathing pattern score thus computed can take a value between zero and one, where a larger score indicates a high likelihood of diaphragmatic breathing and a lower likelihood of chest breathing, and vice versa. A breathing pattern can be classified as diaphragmatic breathing if the breathing pattern score exceeds a threshold, or as chest breathing if the breathing pattern score is below the threshold.

At 540, a physiological event or condition may be detected based at least in part on the classification of the breathing pattern. In an example, an amount of time spent in chest breathing during a specific monitoring or diagnosis time period, and an increase in rapid shallow breathing can be determined if the amount of time spent in chest breathing during a specific monitoring or diagnosis time period exceeds a time threshold or a baseline chest breathing time. A worsening heart failure (WHF) even can be detected based at least on the detected increase in rapid shallow breathing. In another example, an amount of time spent in diaphragmatic breathing or an amount of time spent in chest breathing during a specific monitoring or diagnosis time period can be used to determine a metabolic disorder risk. For example, if the diaphragmatic breathing time falls below a threshold, or the chest breathing time exceeds a time threshold, then a metabolic disorder risk is determined. The patient's body mass index (BMI) may also be considered to determine the risk of metabolic disorder. In some example, the physiological event or condition may be detected based at least in part on a change or a rate of change in breathing pattern from the chest breathing to the diaphragmatic breathing or from the diaphragmatic breathing to the chest breathing. The change or the rate of change in breathing pattern may be detected in response to a physical or emotional trigger event.

At 550, the detection of the physiological event or condition produced at step 540 and/or the classification of the breathing pattern produced at step 530 may be output to a user (e.g., a healthcare provider), or a process such as an instance of a computer program executable in a microprocessor. The detection and classification results may be displayed on a display screen of the user interface 230. The information may be presented in a table, a chart, a diagram, or any other types of textual, tabular, or graphical presentation formats. In an example, alerts, alarms, emergency calls, or other forms of warnings may be generated to inform the system user about the detected physiological event or condition. In some examples, a therapy may be initiated or adjusted based on the detection of the physiological event or condition and/or the classification of the breathing pattern. Examples of the therapy may include electrostimulation therapy delivered to the heart, a nerve tissue, other target tissue, a cardioversion therapy, a defibrillation therapy, or drug therapy.

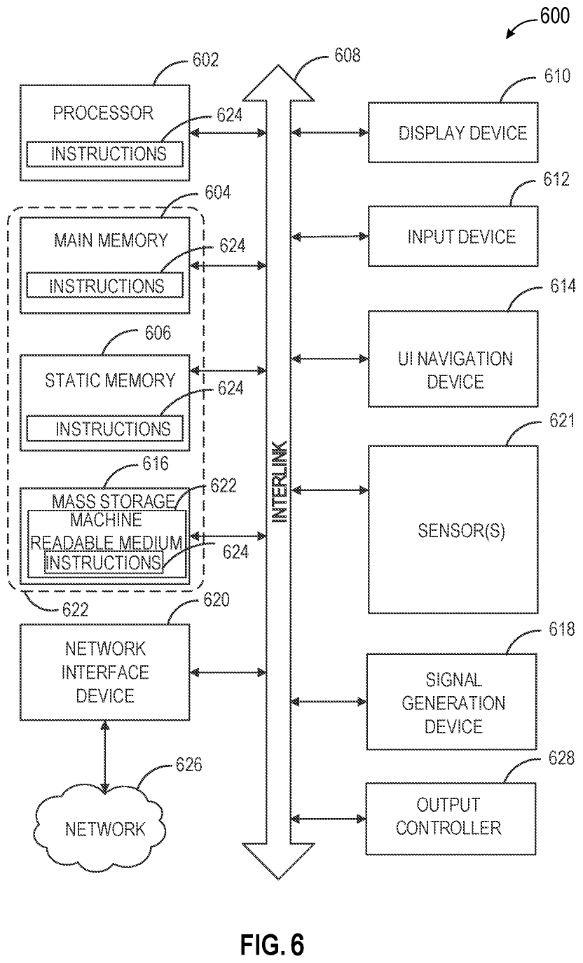

FIG. 6 illustrates generally a block diagram of an example machine 600 upon which any one or more of the techniques (e.g., methodologies) discussed herein may perform. Portions of this description may apply to the computing framework of various portions of an ambulatory device such as the IMD 102 or the WMD 103, or the external device 106, among others.

In alternative embodiments, the machine 600 may operate as a standalone device or may be connected (e.g., networked) to other machines. In a networked deployment, the machine 600 may operate in the capacity of a server machine, a client machine, or both in server-client network environments. In an example, the machine 600 may act as a peer machine in peer-to-peer (P2P) (or other distributed) network environment. The machine 600 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specific operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 600 may include a hardware processor 602 (e.g., a central processing unit (CPU), a graphics processing unit (GPU), a hardware processor core, or any combination thereof), a main memory 604 and a static memory 606, some or all of which may communicate with each other via an interlink (e.g., bus) 608. The machine 600 may further include a display unit 610 (e.g., a raster display, vector display, holographic display, etc.), an alphanumeric input device 612 (e.g., a keyboard), and a user interface (UI) navigation device 614 (e.g., a mouse). In an example, the display unit 610, input device 612 and UI navigation device 614 may be a touch screen display. The machine 600 may additionally include a storage device (e.g., drive unit) 616, a signal generation device 618 (e.g., a speaker), a network interface device 620, and one or more sensors 621, such as a global positioning system (GPS) sensor, compass, accelerometer, or other sensors. The machine 600 may include an output controller 628, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 616 may include a machine readable medium 622 on which is stored one or more sets of data structures or instructions 624 (e.g., software) embodying or utilized by any one or more of the techniques or functions described herein. The instructions 624 may also reside, completely or at least partially, within the main memory 604, within static memory 606, or within the hardware processor 602 during execution thereof by the machine 600. In an example, one or any combination of the hardware processor 602, the main memory 604, the static memory 606, or the storage device 616 may constitute machine-readable media.

While the machine-readable medium 622 is illustrated as a single medium, the term “machine readable medium” may include a single medium or multiple media (e.g., a centralized or distributed database, and/or associated caches and servers) configured to store the one or more instructions 624.

The term “machine readable medium” may include any medium that is capable of storing, encoding, or carrying instructions for execution by the machine 600 and that cause the machine 600 to perform any one or more of the techniques of the present disclosure, or that is capable of storing, encoding or carrying data structures used by or associated with such instructions. Non-limiting machine-readable medium examples may include solid-state memories, and optical and magnetic media. In an example, a massed machine-readable medium comprises a machine readable medium with a plurality of particles having invariant (e.g., rest) mass. Accordingly, massed machine-readable media are not transitory propagating signals. Specific examples of massed machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 624 may further be transmitted or received over a communications network 626 using a transmission medium via the network interface device 620 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as WiFi®, IEEE 802.16 family of standards known as WiMax®), IEEE 802.15.4 family of standards, peer-to-peer (P2P) networks, among others. In an example, the network interface device 620 may include one or more physical jacks (e.g., Ethernet, coaxial, or phone jacks) or one or more antennas to connect to the communications network 626. In an example, the network interface device 620 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 600, and includes digital or analog communications signals or other intangible medium to facilitate communication of such software.

Various embodiments are illustrated in the figures above. One or more features from one or more of these embodiments may be combined to form other embodiments.

The method examples described herein can be machine or computer-implemented at least in part. Some examples may include a computer-readable medium or machine-readable medium encoded with instructions operable to configure an electronic device or system to perform methods as described in the above examples. An implementation of such methods may include code, such as microcode, assembly language code, a higher-level language code, or the like. Such code may include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical-device system, comprising:
a receiver circuit configured to receive respiration information of a patient; and
a breath analyzer circuit configured to:
determine, from the received respiration information, two or more spatial respiration components along respective anatomical axes of the patient, including a first respiration component along an anterior-posterior (AP) axis and one or more second respiration components along a superior-inferior (SI) axis or a left-right (LR) axis; and
classify a breathing pattern as one of either chest breathing or diaphragmatic breathing using the determined two or more spatial respiration components.

2. The medical-device system of claim 1, comprising at least one accelerometer sensor configured to sense multiple directional acceleration signals of chest wall or abdominal movement correlated to respiration, the multiple directional acceleration signals including an anterior-posterior acceleration (XL-AP) signal, a superior-inferior acceleration (XL-SI) signal, and a left-right acceleration (XL-LR) signal,
wherein the breath analyzer circuit is configured to:
determine the two or more spatial respiration components using respective signal metrics of the sensed multiple directional acceleration signals; and
classify the breathing pattern as the diaphragmatic breathing if the signal metric of the XL-AP signal is greater than the signal metric of the XL-SI signal and the signal metric of the XL-LR signal, and to classify the breathing pattern as the chest breathing if the signal metric of the XL-AP signal is no greater than any of the signal metric of the XL-SI signal or the signal metric of the XL-LR signal.

3. The medical-device system of claim 2, wherein to classify the breathing pattern, the breath analyzer circuit is configured to:
calculate a breathing pattern score representing a relative strength of the signal metric of the XL-AP signal with respect to one or more of the signal metric of the XL-SI signal or the signal metric of the XL-LR signal; and
classify the breathing pattern as the diaphragmatic breathing if the breathing pattern score exceeds a threshold, or as the chest breathing if the breathing pattern score is below the threshold.

4. The medical-device system of claim 2, wherein the at least one accelerometer sensor includes a plurality of single-axis accelerometers configured to sense, respectively, the multiple directional acceleration signals of chest wall or abdominal movement.

5. The medical-device system of claim 2, wherein the at least one accelerometer sensor includes a multi-axis accelerometer.

6. The medical-device system of claim 2, wherein the breath analyzer circuit is configured to filter the sensed multiple directional acceleration signals to a respiration frequency range, and to determine the two or more spatial respiration components using the filtered multiple directional acceleration signals.

7. The medical-device system of claim 2, comprising an ambulatory medical device (AMD) configured for abdomen or chest placement, the at least one accelerometer sensor is included in the AMD and configured to sense the multiple directional acceleration signals along respective directions with respect to an orientation of the AMD,
wherein the breath analyzer circuit is configured to determine a spatial relationship between the orientation of the AMD and the anatomical axes of the patient, and to calibrate the multiple directional acceleration signals using the determined spatial relationship.

8. The medical-device system of claim 2, wherein the respective signal metrics of the sensed multiple directional acceleration signals include respective signal amplitudes or signal power within a respiration cycle.

9. The medical-device system of claim 1, wherein the receiver circuit is configured to receive physical activity or posture information of the patient when the respiration information is sensed,
wherein the breath analyzer circuit is configured to determine the two or more spatial respiration components further using the received physical activity or posture information.

10. The medical-device system of claim 1, wherein the breath analyzer circuit is configured to:
generate a trend for each of the two or more spatial respiration components over multiple respiration cycles; and
classify the breathing pattern based at least in part on a comparison of generated trends of the two or more spatial respiration components.

11. The medical-device system of claim 1, further comprising a physiological event detector configured to detect a worsening heart failure event based at least in part on the classified breathing pattern.

12. The medical-device system of claim 1, further comprising a physiological event detector configured to determine a risk of metabolic disorder based at least in part on the classified breathing pattern.

13. The medical-device system of claim 1, further comprising a physiological event detector,
wherein the breath analyzer circuit is configured to, in response to a physical or emotional trigger event, detect a change or a rate of change in breathing pattern from the chest breathing to the diaphragmatic breathing or from the diaphragmatic breathing to the chest breathing,
wherein the physiological event detector is configured to detect a physiological event or condition of the patient based at least in part on the detected change or the detected rate of change in breathing pattern.

14. A method of monitoring respiration using a medical-device system, the method comprising:
receiving respiration information sensed from a patient;
determining, from the received respiration information, two or more spatial respiration components along respective anatomical axes of the patient, including a first respiration component along an anterior-posterior (AP) axis and one or more second respiration components along a superior-inferior (SI) axis or a left-right (LR) axis; and
classifying a breathing pattern as one of either chest breathing or diaphragmatic breathing using the determined two or more spatial respiration components.

15. The method of claim 14, wherein the received respiration information includes multiple directional acceleration signals of chest wall or abdominal movement correlated to respiration, the multiple directional acceleration signals including an anterior-posterior acceleration (XL-AP) signal, a superior-inferior acceleration (XL-SI) signal, and a left-right acceleration (XL-LR) signal,
wherein determining the two or more spatial respiration components includes determining respective signal metrics of the multiple directional acceleration signals,
wherein classifying the breathing pattern incudes classifying the diaphragmatic breathing if the signal metric of the XL-AP signal is greater than the signal metric of the XL-SI signal and the signal metric of the XL-LR signal, and classifying the chest breathing if the signal metric of the XL-AP signal is no greater than any of the signal metric of the XL-SI signal or the signal metric of the XL-LR signal.

16. The method of claim 15, wherein classifying the breathing pattern includes:
calculating a breathing pattern score representing a relative strength of the signal metric of the XL-AP signal with respect to one or more of the signal metric of the XL-SI signal or the signal metric of the XL-LR signal; and
classifying the breathing pattern as the diaphragmatic breathing if the breathing pattern score exceeds a threshold, or as the chest breathing if the breathing pattern score is below the threshold.

17. The method of claim 15, comprising:
sensing the multiple directional acceleration signals using at least one accelerometer sensor included in an ambulatory medical device (AMD) configured for abdomen or chest placement, the multiple directional acceleration signals being sensed along respective directions with respect to an orientation of the AMD;
determining a spatial relationship between the orientation of the AMD and the anatomical axes of the patient; and
calibrating the multiple directional acceleration signals using the determined spatial relationship.

18. The method of claim 15, wherein the respective signal metrics of the multiple directional acceleration signals include respective signal amplitudes or signal power within a respiration cycle.

19. The method of claim 14, comprising detecting a worsening heart failure event or a risk of metabolic disorder based at least in part on the classified breathing pattern.

20. The method of claim 14, further comprising:
in response to a physical or emotional trigger event, detecting a change or a rate of change in breathing pattern from the chest breathing to the diaphragmatic breathing or from the diaphragmatic breathing to the chest breathing; and
detecting a physiological event or condition of the patient based at least in part on the detected change or the detected rate of change in breathing pattern.
